(19) United States
(12) Patent Application Publication
(10) Pub. No.: US 2018/0259497 A1
(43) Pub. Date: Sep. 13, 2018

(54) INFORMATION PRESENTATION SYSTEM AND METHOD FOR CONTROLLING THE INFORMATION PRESENTATION SYSTEM

(71) Applicant: PANASONIC INTELLECTUAL PROPERTY MANAGEMENT CO., LTD., Osaka (JP)

(72) Inventors: Kazuhiro OCHI, Kyoto (JP); Hiroyuki KAIZO, Shiga (JP); Yutaka YAMADA, Osaka (JP); Daisuke MORIKAWA, Kyoto (JP)

(21) Appl. No.: 15/911,625

(22) Filed: Mar. 5, 2018

(30) Foreign Application Priority Data
Mar. 9, 2017 (JP) 2017-045135

(51) Int. Cl. G01N 33/02; G01G 19/414

(52) U.S. Cl. CPC G01N 33/02; G01G 19/4146

(57) ABSTRACT

An information presentation system according to the present disclosure includes: an analyzer that makes an analysis of food and outputs an analysis value indicating a result of the analysis; a generator that generates information on at least one of food ingestion by a user and calorie consumption by the user based on the analysis value output by the analyzer; and a presentation unit that presents the information generated by the generator. In this way, the information presentation system of the present disclosure can improve display of the analysis result of the food analyzer.

### BACKGROUND

### 1. Technical Field

The present disclosure relates to an information presentation system and to a method for controlling the information presentation system.

### 2. Description of the Related Art

Unexamined Japanese Patent Publication No. 2015-118008 proposes a food analyzer that analyzes calories or nutrients of food which is an object to be measured, and displays an analysis result in a mode of being easily recognizable by a viewer.

### SUMMARY

However, in the food analyzer that displays the analysis result of the food, it has been required that the display of the analysis result be further improved.

An information presentation system according to an aspect of the present disclosure includes: an analyzer that makes an analysis for food and outputs an analysis value indicating a result of the analysis; a generator that generates information on at least one of food ingestion by a user and calorie consumption by the user based on the analysis value output by the analyzer; and a presentation unit that presents the information generated by the generator.

Note that these general or specific aspects may be implemented by a system, a method, an integrated circuit, a computer program, or a recording medium such as a computer-readable CD-ROM, or may be implemented by arbitrary combinations of the system, the method, the integrated circuit, the computer program and the recording medium.

The information presentation system of the present disclosure can improve the display of the analysis result of the food analyzer.

### BRIEF DESCRIPTION OF THE DRAWINGS

FIG. 1 is a block diagram showing a configuration of an information presentation system in a first exemplary embodiment;

FIG. 2 is a schematic diagram showing an example of a detailed configuration of an analyzer in the first exemplary embodiment;

FIG. 3 is a schematic diagram showing an example of a detailed configuration of a light receiver in the first exemplary embodiment;

FIG. 4 is an explanatory view showing an example of a distribution image of calories, which is output by the analyzer in the first exemplary embodiment;

FIG. 5 is an explanatory diagram showing an example of a distribution image of components, which is output by the analyzer in the first exemplary embodiment;

FIG. 6 is a flowchart showing a method for controlling the information presentation system in the first exemplary embodiment;

FIG. 7 is a block diagram showing a configuration of an information presentation system in a second exemplary embodiment;

FIG. 8 is an explanatory view showing a recipe to be presented by the information presentation system in the second exemplary embodiment;

FIG. 9 is a block diagram showing a configuration of an information presentation system in a third exemplary embodiment;

FIG. 10 is an explanatory view showing an example of eating-out data in the third exemplary embodiment;

FIG. 11 is an explanatory view showing an example of information to be presented by a presentation unit in the third exemplary embodiment;

FIG. 12 is a block diagram showing a configuration of an information presentation system in a fourth exemplary embodiment;

FIG. 13 is an explanatory view showing an example of menu data in the fourth exemplary embodiment;

FIG. 14 is an explanatory view showing a first example of a presentation by the information presentation system in the fourth exemplary embodiment;

FIG. 15 is an explanatory view showing a second example of the presentation by the information presentation system in the fourth exemplary embodiment;

FIG. 16 is a block diagram showing a configuration of an information presentation system in a fifth exemplary embodiment;

FIG. 17 is a block diagram showing a configuration of a presentation unit in a sixth exemplary embodiment;

FIG. 18 is a block diagram showing a first example of an image to be presented by the presentation unit in the sixth exemplary embodiment; and

FIG. 19 is a block diagram showing a second example of the image to be presented by the presentation unit in the sixth exemplary embodiment.

### DETAILED DESCRIPTION

An information presentation system according to an aspect of the present disclosure includes: an analyzer that makes an analysis for food and outputs an analysis value indicating a result of the analysis; a generator that generates information on at least one of food ingestion by a user and calorie consumption by the user based on the analysis value output by the analyzer; and a presentation unit that presents the information generated by the generator.

In accordance with the above-described aspect, the information presentation system generates and presents not only information on calories of food that is an object to be analyzed by the analyzer but also the information on at least one of the food ingestion by the user and the calorie consumption by the user, the information being generated based on the calories of the food, and the like. As described above, the information presentation system can improve display of the result of the analysis by the analyzer.

For example, the analyzer may analyze food cooked in accordance with a recipe of a cooking menu, and may output, as the analysis value, at least one of calories and nutrients contained in the food, the generator may generate information indicating at least one of calories and nutrients of the cooking menu based on at least one of the calories and the nutrients contained in the food and output by the analyzer, and the presentation unit may present, together with the recipe, the information indicating at least one of the calories and the nutrients, the information being generated by the generator.

In accordance with the above-described aspect, the information presentation system presents the information, which indicates the calories or the nutrients, together with the recipe of the cooking menu. Accordingly, the user can grasp in advance the calories or nutrients of the cooking menu in case of cooking in accordance with the presented recipe, and the convenience for the user is enhanced.

For example, the information presentation system may further include a storage that stores recipes of one or more cooking menus, wherein the analyzer may analyze food cooked in accordance with a recipe of a cooking menu included in the one or more cooking menus, and may output, as the analysis value, at least one of calories and nutrients contained in the food, the generator may generate information indicating at least one of calories and nutrients of the cooking menu based on at least one of the calories and the nutrients contained in the food and output by the analyzer, and may cause the storage to store the information generated, and the presentation unit may present, together with a recipe of a target cooking menu searched by the user among the one or more cooking menus stored in the storage, information indicating at least one of calories and nutrients of the target cooking menu stored in the storage.

In accordance with the above-described aspect, the information presentation system stores the recipes of the one or more cooking menus and the calories or nutrients of the cooking menus, and provides the user with the recipe, calories or nutrients of the cooking menu that satisfies the search condition given by the user. Hence, based on the more specific configuration, the display of the analysis result by the analyzer can be improved, and the convenience for the user can be enhanced.

For example, the analyzer may analyze food taken in by the user, and may output, as the analysis value, at least one of calories and nutrients contained in the food, and the generator may generate information on an eating-out menu that the user should eat, based on at least one of the calories and the nutrients output by the analyzer, and on eating-out menu data including at least one of ingestion of food and consumption of calories when the user eats an eating-out menu.

In accordance with the above-described aspect, the information presentation system presents the calories and nutrients of the menu offered by the restaurant. Therefore, not only the average persons but also persons who need dietary restrictions are facilitated to determine the restaurant and the menu. Hence, the display of the analysis result by the analyzer can be improved, and the convenience for the user can be enhanced. Moreover, the user is facilitated to eat out, and therefore, for the restaurants, there are advantages that the number of customers increases and that the sales increase.

For example, the analyzer may analyze food taken in by the user, and may output, as the analysis value, at least one of calories and nutrients contained in the food, the generator may generate information on a cooking menu that the user should eat, based on at least one of the calories and the nutrients output by the analyzer, and on cooking data including at least one of calories and nutrients of ingredients required to cook a plurality of the cooking menus, and the information on the cooking menu may include at least the ingredients required to cook the cooking menu.

In accordance with the above-described aspect, the information presentation system provides the recipe of the meal menu, which the user should eat, based on the foods taken in by the user. Hence, the user can easily acquire the recipe required to cook the meal menu.

For example, the information presentation system may further include an order processing unit that performs order processing for at least one of the ingredients required to cook the cooking menu and the cooking menu already cooked, the ingredients being included in the information on the cooking menu, the information being generated by the generator.

In accordance with the above-described aspect, based on the foods taken in by the user, the information presentation system provides the recipe of the meal menu, which the user should eat, and in addition, orders the ingredients required to cook the meal menu. Hence, the user can easily acquire the recipe required to cook the meal menu, and in addition, the ingredients required to cook the meal menu.

For example, the analyzer may analyze food taken in by the user, and may output, as the analysis value, at least one of calories and nutrients contained in the food, and the generator may generate at least one of information on an exercise menu that the user should do in order to achieve a target body shape, and information on food that the user should take in based on a current body shape and target body shape of the user and on at least one of the calories and the nutrients output by the analyzer.

In accordance with the above-described aspect, the information presentation system provides the exercise menu which the user should do, or the cooking menu which the user should eat. The user exercises in accordance with the provided exercise menu or has a meal in accordance with the presented cooking menu, and can thereby bring the body shape of the user him/herself closer to the target body shape, or can maintain the target body shape.

For example, the presentation unit may include a performance production unit that generates information having a performance production effect for the user based on the information indicating at least one of the calories and the nutrients, the information being generated by the generator, and may present the information having the performance production effect for the user, the information being generated by the performance production unit.

In accordance with the above-described aspect, the information presentation system presents the information produced so that the user has some kind of feeling. This information is generated based on the information on the numeric value indicating the calories or nutrients of the food, the ingestion of the food by the user, and the consumption of the calories by the user. In this way, the user is prevented from losing interest in the provided information, and the user can obtain the information by continuously using the information presentation system.

For example, the performance production unit may generate benefit information different depending on at least one of a specific ratio and a random element when the presentation unit presents the information having the performance production effect for the user, and the presentation unit may present the benefit information.

In accordance with the above-described aspect, the information presentation system presents the benefit information at a predetermined ratio. When the information is provided from the information presentation system, the user sometimes obtains the benefit information, and sometimes does not obtain the benefit information. In this way, the user uses the information presentation system while expecting that he/she can obtain the benefit information. In this way, the user is prevented from losing interest in the provided information, and the user can obtain the information by continuously using the information presentation system.

For example, the analyzer may analyze at least one of components of the food, the components including calories, carbohydrate, lipid, protein, dietary fiber, and glucide.

In accordance with the above-described aspect, the information presentation system can specifically present the information based on an analysis value of the components of the food, which include calories, carbohydrate, lipid, protein, dietary fiber and glucide.

A method for controlling an information presentation system according to an aspect of the present disclosure may include: an analysis step of making an analysis for food and outputs an analysis value indicating a result of the analysis; a generation step of generating information on at least one of food ingestion by a user and calorie consumption by the user based on the analysis value output in the analysis step; and a presentation step of presenting the information generated in the generation step.

In accordance with the above-described aspect, a similar effect to that of the above-described information presentation system is exerted.

Note that these general or specific aspects may be implemented by a system, a method, an integrated circuit, a computer program, or a recording medium such as a computer-readable CD-ROM, or may be implemented by arbitrary combinations of the system, the method, the integrated circuit, the computer program and the recording medium.

Hereinafter, a specific description will be given of exemplary embodiments with reference to the drawings.

Note that all of the exemplary embodiments to be described below represent general or specific examples. Numeric values, shapes, ingredients, constituents, arrangement positions and connection modes of the constituents, steps, orders of the steps, and the like, which are shown in the following exemplary embodiments, are merely examples, and are not intended to limit the present disclosure. Moreover, among the constituents in the exemplary embodiments to be described below, constituents which are not described in the independent claims showing the top level concept are described as arbitrary constituents.

### First Exemplary Embodiment

In the present exemplary embodiment, a description will be given of information presentation system 1 that improves the display of the analysis result of the food analyzer.

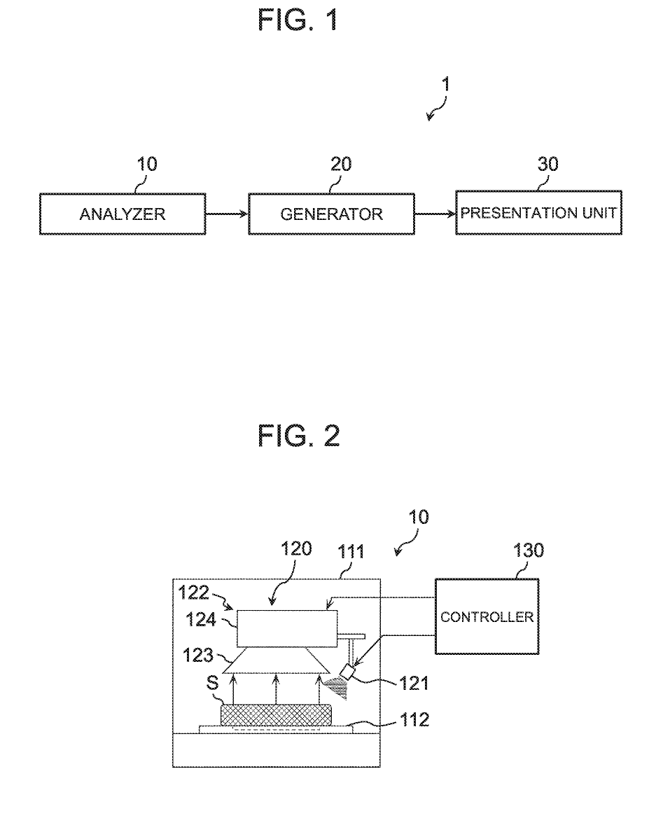

FIG. 1 is a block diagram showing a configuration of information presentation system 1 in the present exemplary embodiment.

As shown in FIG. 1, information presentation system 1 includes analyzer 10, generator 20 and presentation unit 30.

Analyzer 10 is a food analyzer that makes an analysis for food and outputs an analysis value indicating a result of the analysis. Specifically, analyzer 10 analyzes calories or nutrients of the food as an object to be analyzed. The nutrients of the food include components as carbohydrates, lipids, proteins, dietary fibers or glucides. Note that a configuration, food analysis principle and the like of analyzer 10 are not particularly limited. Examples of the configuration and analysis principle of analyzer 10 will be described later in detail.

Generator 20 is a processing unit that generates information on food ingestion by the user or calorie consumption by the user based on the analysis value output by analyzer 10. Generator 20 may be implemented in such a manner that a processor executes a program, or may be implemented by dedicated hardware.

Presentation unit 30 is a processing unit that presents the information generated by generator 20. A mode in which presentation unit 30 presents the information can include a variety of options. For example, the mode can include such options as display of an image, generation of a sound, and a generation of an odor, but is not limited to these. Presentation unit 30 can be implemented by a display, a speaker, an odor generator or the like, a control circuit for these, and the like. The display may be a display provided on a part of a housing of analyzer 10 (for example, a front panel), or may be a display screen of a communication terminal communicable with analyzer 10.

Hereinafter, the examples of the configuration and analysis principle of analyzer 10 will be described in detail with reference to FIG. 2 to FIG. 5.

The configuration of analyzer 10 will be described with reference to FIG. 2.

Analyzer 10 includes housing 111, table 112, measurement unit 120 and controller 130.

Table 112 and measurement unit 120 are disposed in an inside of housing 111. Object to be measured S is disposed on table 112. Note that analyzer 10 can analyze one or plural foods as object to be measured S. Moreover, analyzer 10 can also analyze food, which is contained in a container or the like, as object to be measured S.

Measurement unit 120 includes: light source 121 that irradiates object to be measured S, which is placed on table 112; and light receiver 122 that receives light reflected from object to be measured S.

Light source 121 is disposed at a position from which a whole of object to be measured S can be irradiated. Light applied from light source 121 includes at least a partial wavelength of near infrared light with a wavelength range of 700 nm to 2500 nm, inclusive. As light source 121, for example, there is used a halogen lamp, an LED (Light Emitting Diode), a laser, or the like.

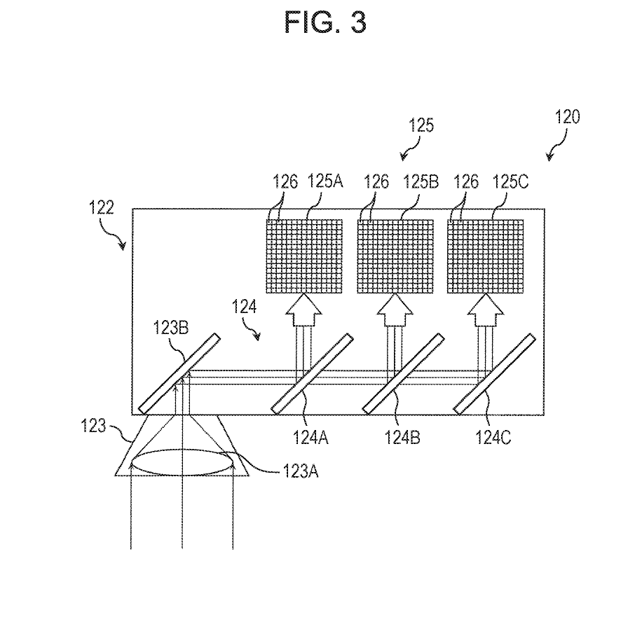

FIG. 3 is a schematic diagram showing an example of a detailed configuration of light receiver 122 in the present exemplary embodiment.

Light receiver 122 includes light condenser 123, specific wavelength reflector 124, and light receiving sensor 125. Light receiver 122 is disposed at a position of being capable of receiving light reflected from object to be measured S. It is preferable that a distance between light receiver 122 and object to be measured S be small.

Light condenser 123 includes condenser lens 123A and reflector 123B.

Specific wavelength reflector 124 includes reflectors 124A, 124B and 124C. Reflectors 124A, 124B and 124C are composed as wavelength selective filters which reflect only light with specific wavelengths and transmit light with other wavelengths therethrough. Reflector 124A reflects only light with a first specific wavelength, and transmits light with other wavelengths therethrough. Reflector 124B reflects only light with a second specific wavelength, and transmits light with other wavelengths therethrough. Reflector 124C reflects only light with a third specific wavelength, and transmits light with other wavelengths therethrough.

The first to third specific wavelengths are determined by experiment based on spectral information on a plurality of foods of which components are already known. Specifically, wavelengths which satisfactorily reflect proportions of specific components in the plurality of foods are determined as the specific wavelengths based on relationships between the proportions and absorbances of the specific components.

As the first specific wavelength, a wavelength that satisfactorily correlates with protein as a component is adopted. For example, as the first specific wavelength, a wavelength of approximately 910 nm can be adopted. As the second specific wavelength, a wavelength that satisfactorily correlates with lipid as a component is adopted. For example, as the second specific wavelength, a wavelength of approximately 930 nm can be adopted. As the third specific wavelength, a wavelength that satisfactorily correlates with carbohydrate as a component is adopted. For example, as the third specific wavelength, a wavelength of approximately 980 nm can be adopted.

The light receiving sensor 125 includes a plurality of light receiving elements 126 arranged in a grid pattern. Light receiving sensor 125 is composed as a so-called image sensor. As light receiving elements 126, for example, such elements are usable, which use silicon and indium/gallium/arsenic and can convert light into electrical signals. Here, silicon and indium/gallium/arsenic have wide sensitivity in a near-infrared region.

With reference to FIG. 3, a description will be given of optical paths and operations of measurement unit 120 at a time of analyzing object to be measured S.

Upon receiving an instruction to start analysis of food from the user, controller 130 controls measurement unit 120 to start to analyze object to be measured S. For example, the instruction can be made by pressing a button, receiving predetermined information, and so on.

Controller 130 controls light source 121 (refer to FIG. 2) to momentarily irradiate object to be measured S with light including near infrared light. Light scattered and reflected from object to be measured S is condensed by condenser lens 123A, and as parallel light, enters reflector 123B.

The light that has entered reflector 123B is reflected toward specific wavelength reflector 124, and enters specific wavelength reflector 124.

With regard to the light that has entered specific wavelength reflector 124, in reflector 124A, only light with the first specific wavelength is reflected toward light receiving sensor 125A. Light with the rest of the wavelengths passes through reflector 124A, and enters reflector 124B.

With regard to the light that has entered reflector 124B, in reflector 124B, only light with the second specific wavelength is reflected toward light receiving sensor 125B. Light with the rest of the wavelengths passes through reflector 124B, and enters reflector 124C.

With regard to the light that has entered reflector 124C, in reflector 124C, only light with the third specific wavelength is reflected toward light receiving sensor 125C. Light with the rest of the wavelengths passes through reflector 124C.

Rays included in the light that goes toward respective light receiving sensors 125A to 125C maintain a positional relationship among rays included in the light immediately after being scattered and reflected from object to be measured S. Therefore, outputs of respective light receiving elements 126 reflect the protein, the lipid and the carbohydrate in a part of object to be measured S. That is, respective light receiving elements 126 output signals, which reflect the protein, the lipid and the carbohydrate in the part (hereinafter, “area to be measured”) of object to be measured S, to controller 130 (refer to FIG. 2). Here, the part of object to be measured S is a starting point of the rays to be received by the light receiving elements 126.

Controller 130 calculates the proportion and quantity of the protein based on the outputs of light receiving elements 126 of light receiving sensor 125A and a prestored relational expression. Controller 130 calculates the proportion and quantity of the lipid based on the outputs of light receiving elements 126 of light receiving sensor 125B and a prestored relational expression. Controller 130 calculates the proportion and quantity of the carbohydrate based on the outputs of light receiving elements 126 of light receiving sensor 125C and a prestored relational expression. Note that the respective relational expressions can be determined by using an analysis method such as a PLS (Partial Least Squares) method, linear multiple regression and a neural net, for example, based on relationships between absorbances (quantities of light) in the respective wavelengths and proportions of the respective components in the plurality of foods of which components are already known.

Calories of food are determined by multiplying a quantity of protein, a quantity of lipid and a quantity of carbohydrate individually by calorie coefficients, and by adding the multiplied quantities to one another. Therefore, based on the quantity of the protein, the quantity of the lipid and the quantity of the carbohydrate for each of the areas to be measured, controller 130 calculates the calories for each area to measured. Note that, in each area to be measured, the proportion of the protein, the quantity of the protein, the proportion of the lipid, the quantity of the lipid, the proportion of the carbohydrate, the quantity of the carbohydrate and the calories are also referred to as “partial nutrition information”.

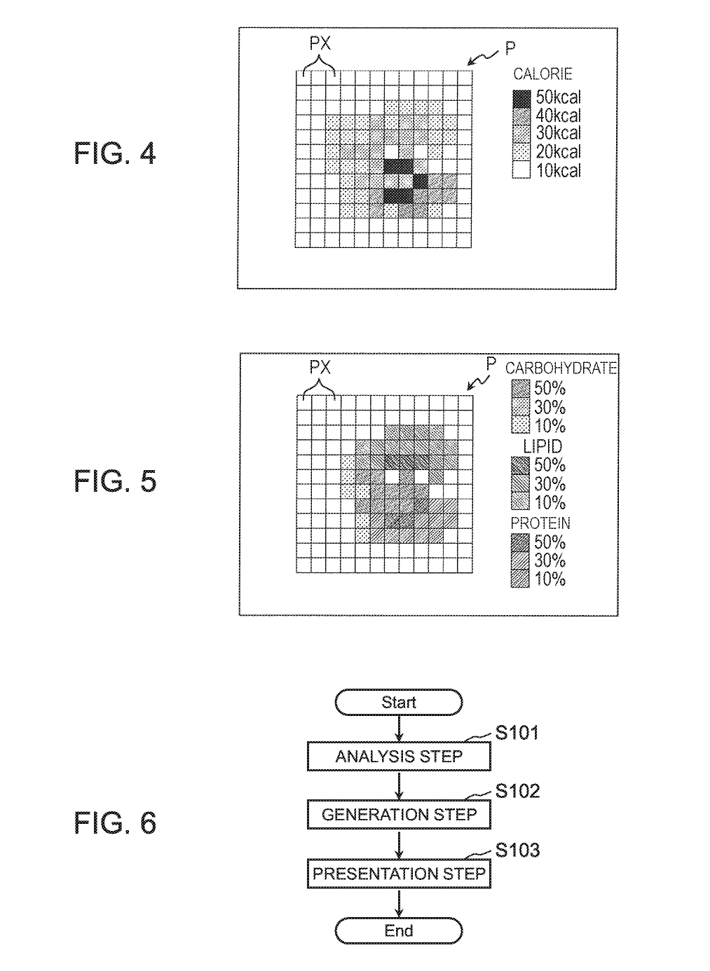

Positions of the light receiving elements 126 in light receiving sensor 125 correspond to a position of the area to be measured. Controller 130 combines the positions of light receiving elements 126 and the partial nutrition information calculated based on the outputs of light receiving elements 126, and generates and outputs distribution image information. Specifically, controller 130 generates distribution image information for displaying the calories and distribution image information for displaying the components as the distribution image information. A description will be given of distribution image P, which corresponds to the distribution image information to be generated by controller 130, with reference to FIGS. 4 and 5. In FIGS. 4 and 5, densities of display colors are represented by point densities of point hatching. Moreover, in FIG. 5, differences of the display colors are represented by whether or not line hatching exists and by directions of lines.

FIG. 4 is an explanatory view showing an example of distribution image P of the calories, which is output by analyzer 10 in the present exemplary embodiment.

As shown in FIG. 4, distribution image P of the calories is divided into a plurality of partial regions PX. Single partial region PX displays single partial nutrition information. A positional relationship among respective partial regions PX maintain a positional relationship among the areas to be measured, which correspond to the partial nutrition information displayed on respective partial regions PX.

The densities of the display colors of respective partial regions PX are allowed to correspond to magnitudes of the calories, whereby distribution image P of the calories visually expresses the magnitudes of the calories of respective partial regions PX.

FIG. 5 is an explanatory diagram showing an example of distribution image P of components, which is output by analyzer 10 in the present exemplary embodiment.

As shown in FIG. 5, distribution image P of the components visually expresses components, which have largest proportions among the protein, the lipid and the carbohydrates in respective partial regions PX, by using display colors different for each of the components. Moreover, densities of the display colors in respective partial regions PX are allowed to correspond to the proportions of the components, whereby distribution image P visually expresses the ratio of the components of respective partial regions PX.

Note that analyzer 10 is a stationary device having an internal space in which it is possible to place object to be measured S, in other words, is a device like a microwave oven or a toaster in terms of configuration, but is not limited to this. For example, analyzer 10 may be a device that emits light toward object to be measured S placed outside analyzer 10 and receives light reflected by object to be measured S. In other words, analyzer 10 may be a device like a cellular phone, a smart phone or a digital camera in terms of configuration. Note that analyzer 10 may be implemented as a single function of a microwave oven, a toaster, a cellular phone, a smart phone or a digital camera.

A description will be given of operations of information presentation system 1 configured as described above.

FIG. 6 is a flowchart showing a method for controlling information presentation system 1 in the present exemplary embodiment.

In Step S101 (analysis step), analyzer 10 makes an analysis for food, and outputs an analysis value indicating a result of the analysis.

In Step S102 (generation step), generator 20 generates information on food ingestion by the user or calorie consumption by the user based on the analysis value output in the analysis step.

In Step S103 (presentation step), presentation unit 30 presents the information generated in the generation step.

In this way, information presentation system 1 can improve the display of the result of the analysis by analyzer 10. More specifically, information presentation system 1 generates and presents not only information on calories of the food which is an object to be analyzed by analyzer 10 but also the information on the food ingestion by the user or the calorie consumption by the user, the information being generated based the calories of the food, and the like. As described above, information presentation system 1 can improve the display of the result of the analysis by analyzer 10.

Specifically, information presentation system 1 is applicable to purposes such as presentation of cooking recipes, presentation of a menu for eating out, presentation of a menu of meals at home, and presentation of a menu of meals for managing a body shape of a user. These will be described in detail in the exemplary embodiments to be described later.

Moreover, information presentation system 1 is applicable to purposes such as nutrition management for residents in nursing home, nutrition management for students in school facilities, and provision of advice on life habits in medical institutions. Specifically, by analyzer 10, information presentation system 1 analyzes each of still uneaten food and food left uneaten, and analyzes calories and nutrients, which are taken in by a person from meals. Then, by generator 20, information presentation system 1 generates a cooking menu that enables the person to take in calories and nutrients, which compensate for lacks with respect to predetermined quantities of calories and nutrients, the quantities being to be taken in by the person. Then, by presentation unit 30, information presentation system 1 presents the generated cooking menu.

In the following respective exemplary embodiments, a description will be given of typical specific examples of information presentation system 1.

### Second Exemplary Embodiment

In the present exemplary embodiment, a description will be given of an information presentation system that improves the display of the analysis result of the food analyzer and provides the cooking recipe. For example, this information presentation system can be implemented by using a so-called recipe site that is a recipe-providing website prepared on the Internet and by using a communication terminal that acquires information from the recipe site and presents the information to a user.

A conventional recipe site has a plurality of cooking recipes. Then, based on a search by the user by using search conditions regarding a name, ingredients, and the like of a dish, the conventional recipe site provides a recipe that matches the search condition. For example, when the user wishes to search for a recipe of stew using chicken, if the user makes the search by using a search condition as “chicken” and “stew”, then the recipe site provides the communication terminal with a recipe of “chicken stew” among recipes owned by the recipe site itself, and presents the recipe to the user.

The cooking recipes owned by the conventional recipe site include recipes provided by a variety of persons including general users. Accordingly, in some cases, the cooking recipes may not include information on calories and nutrients of the relevant dish. Moreover, even when the cooking recipes include the information described above, accuracy or precision of values may be unknown in some cases.

Meanwhile, if the calories and nutrients of the dish are provided from the recipe site, then the user will be able to select a recipe based on the provided calories and nutrients. Moreover, convenience for the user is enhanced if the user who acquires the recipe from the recipe site can search for the recipe by using the information on the calories, nutrients and the like of the dish. Furthermore, if calories and nutrients of dishes cooked in the past with reference to the relevant recipe are provided, then there is an advantage that statistical treatment and the like enhance the accuracy and precision of the values.

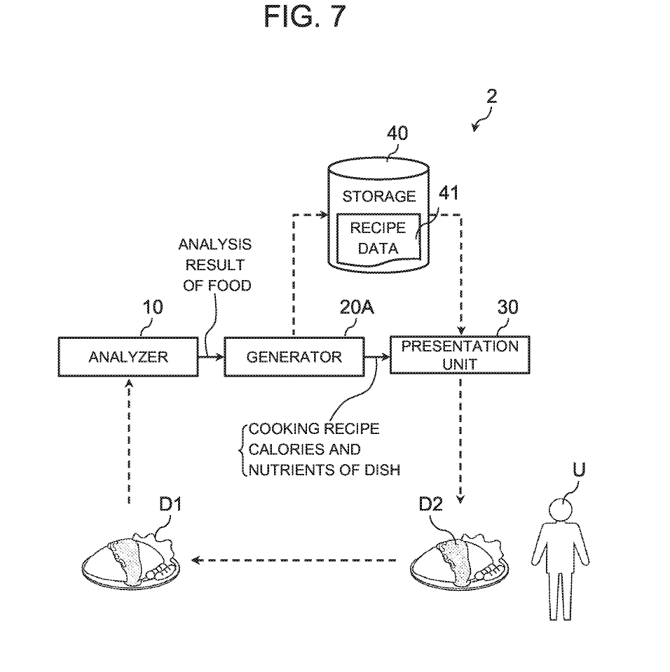

FIG. 7 is a block diagram showing a configuration of information presentation system 2 in the present exemplary embodiment.

As shown in FIG. 7, information presentation system 2 includes analyzer 10, generator 20A, presentation unit 30, and storage 40.

Analyzer 10 is a food analyzer that makes an analysis for dish D1 as food and outputs an analysis value indicating a result of the analysis. This analyzer 10 is similar to analyzer 10 in the first exemplary embodiment. Analyzer 10 analyzes dish D1 cooked in accordance with a recipe of a cooking menu, and outputs calories or nutrients, which are contained in dish D1, as an analysis value. Note that the expression “calories or nutrients” means at least one of the calories and the nutrients. The same applies hereinafter.

Based on the calories or the nutrients, which are contained in dish D1 and are output by analyzer 10, generator 20A generates information indicating the calories or nutrients of the cooking menu. Generator 20A is one of specific configurations of generator 20.

Presentation unit 30 presents the information, which indicates the calories or the nutrients and is generated by generator 20A, together with the cooking recipe.

An example of a more specific configuration will be described below.

Information presentation system 2 may further include storage 40.

Storage 40 is a storage device that stores recipe data 41 including recipes of one or more cooking menus. Storage 40 is formed of a volatile memory or a nonvolatile memory.

Moreover, the recipe of dish D1 that is an object to be analyzed by analyzer 10 may be a recipe included in the one or more cooking menus stored in storage 40.

Furthermore, generator 20A may store the information, which is generated thereby and indicates the calories or nutrients of the cooking menu, in storage 40.

Moreover, presentation unit 30 may present information, which indicates calories or nutrients of such a target cooking menu among the one or more cooking menus stored in storage 40, together with a recipe of the target cooking menu. Here, this recipe is searched by the user, and the calories or the nutrients of the target cooking menu are also stored in storage 40.

User U can prepare dish D2 by cooking in accordance with the recipe presented by presentation unit 30. In this way, together with the searched recipe of the cooking menu, the user can grasp the calories or nutrients of the cooking menu, and can cook.

Note that, by analyzer 10, user U can analyze dish D2 by using dish D2 as dish D1 In this way, an analysis result of the dish cooked by the user in accordance with the recipe provided by information presentation system 2 can be reflected on the recipe owned by storage 40 of information presentation system 2. When analysis results of dishes cooked by a plurality of users U are provided to storage 40, statistical treatment for obtaining an average value, median or modal value of these, and so on may be carried out. In this way, the precision of the calories or nutrients of the cooking menu stored in storage 40 can be enhanced.

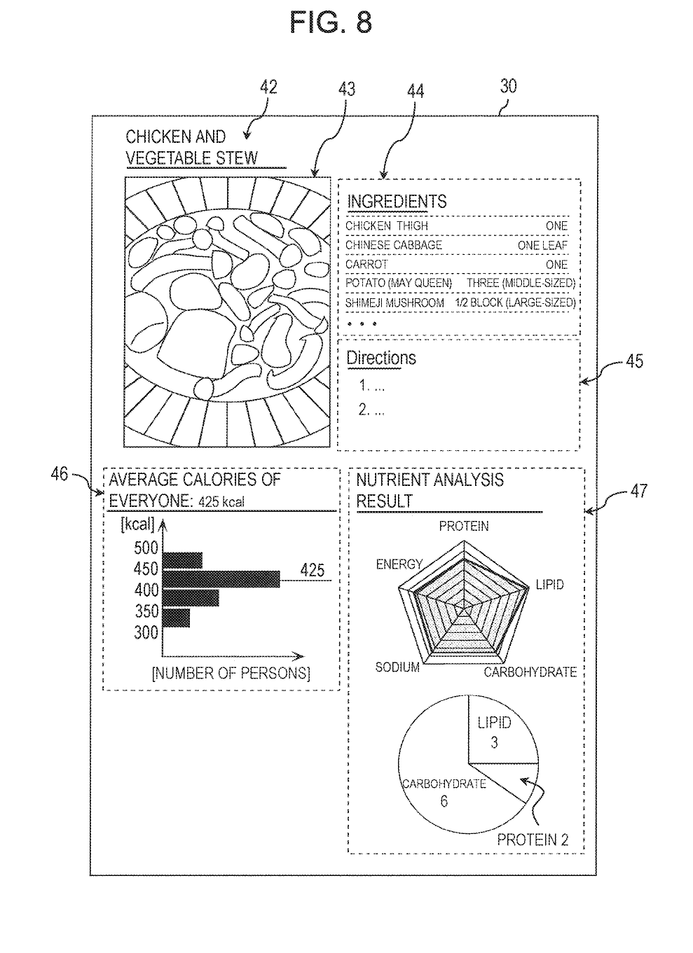

FIG. 8 is an explanatory view showing the recipe to be presented by information presentation system 2 in the present exemplary embodiment. As an example, this recipe is displayed on a display as presentation unit 30.

The recipe includes cooking menu 42, exterior view 43, ingredients 44, direction 45, calorie information 46, and nutrient information 47.

Cooking menu 42, exterior view 43, ingredients 44 and directions 45 are a character string indicating a name, a picture or illustration of an exterior appearance, ingredients, and a direction, respectively with regard to the cooking menu. Cooking menu 42, exterior view 43, ingredients 44 and directions 45 are equivalent to those presented by the conventional recipe site.

Calorie information 46 is information indicating calories obtained in such a manner that analyzer 10 analyzed the dishes cooked in the past by the plurality of users U in accordance with the recipe. For example, calorie information 46 includes a histogram of the calories of the dishes cooked in the past by the plurality of users U in accordance with the recipe, and an average value of the calories described above.

Nutrient information 47 is information indicating nutrients obtained in such a manner that analyzer 10 analyzed the dishes cooked in the past by the plurality of users U in accordance with the recipe. For example, nutrient information 47 includes display of nutrition components (energy, protein, lipid, carbohydrate, sodium) or display of three major nutrients (carbohydrate, protein, and lipid) with regard to the dishes cooked in the past by the plurality of users U in accordance with the recipe.

In this way, the user will be able to select the recipe based on the provided calories and nutrients. Moreover, the user who acquires the recipe from the recipe site can search for the recipe by using the information on the calories, nutrients and the like of the dish, and convenience for the user is enhanced.

If a provider of the recipe site measures the calories and nutrients of the dishes and posts the measured calories and nutrients, then the provider of the recipe site will carry out detailed nutrition analysis individually for the recipes owned by the recipe site, and a workload of the provider can be enormous. As described above, the users provide the recipe site with the calories and nutrients of the dishes cooked thereby, whereby there is an advantage that the workload of the provider of the recipe site can be reduced.

As described above, information presentation system 2 of the present exemplary embodiment presents the information, which indicates the calories or the nutrients, together with the recipe of the cooking menu. Accordingly, the user can grasp in advance the calories or nutrients of the cooking menu in case of cooking in accordance with the presented recipe, and the convenience for the user is enhanced.

Moreover, information presentation system 2 stores the recipes of the one or more cooking menus and the calories or nutrients of the cooking menus, and provides the user with the recipe, calories or nutrients of the cooking menu that satisfies the search condition given by the user. Hence, based on the more specific configuration, the display of the analysis result by the analyzer can be improved, and the convenience for the user can be enhanced.

### Third Exemplary Embodiment

In the present exemplary embodiment, a description will be given of an information presentation system that improves the display of the analysis result of the food analyzer and provides a recommended eating-out menu. For example, this information presentation system can be implemented by using a so-called restaurant search site as a website, which is prepared on the Internet and provides restaurants (also called eating shops) and eating-out menus, and by using a communication terminal that acquires information from the restaurant search site and presents the information to a user.

A conventional restaurant search site owns a plurality of the restaurants and menus or courses, which are provided by the restaurants. Then, based on a search by the user by using search conditions regarding a name, expense and the like of a dish, the conventional restaurant search site provides a menu or a course, which matches the search condition. For example, when the user wishes to search for a restaurant that provides tofu (bean curd) hamburger steak, if the user makes the search by using a search condition as “tofu hamburger steak”, the restaurant search site provides information regarding a restaurant, which provides “tofu hamburger steak”, from among the owned restaurants and menus, and presents the information to the user.

The restaurants and the menus, which are owned by the conventional restaurant search site, are not supposed to be offered to those who need dietary restrictions due to illness or a constitutional predisposition though calorie information and the like are sometimes posted therein to some extent while being supposed to be offered to average persons. Therefore, it is sometimes unknown whether or not the menus offered by the restaurants are usable for those who are on diet.

Meanwhile, if the calories and nutrients of the menus offered by the restaurants are provided by the restaurant search site, then not only the average persons but also those who need the dietary restrictions are facilitated to determine the restaurant and the menu, and are consequently facilitated to eat out. Also for the restaurants, there are advantages that a number of customers increases and that sales increase.

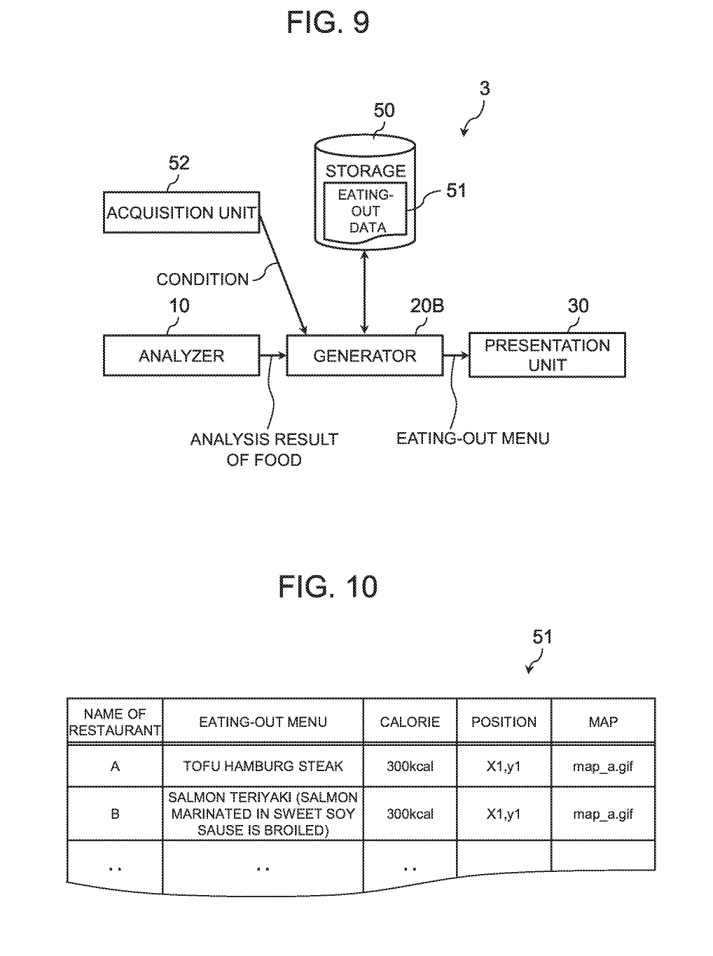

FIG. 9 is a block diagram showing a configuration of information presentation system 3 in the present exemplary embodiment.

As shown in FIG. 9, information presentation system 3 includes analyzer 10, generator 20B, presentation unit 30, storage 50 and acquisition unit 52.

Analyzer 10 is a food analyzer that makes an analysis for food taken in by the user and outputs calories or nutrients, which are contained in the food, as an analysis value. This analyzer 10 is similar to analyzer 10 in the first exemplary embodiment. Analyzer 10 analyzes at least one of the calories or the nutrients, which the user takes in from meals every day.

Storage 50 is a storage device that stores eating-out data 51 including one or more eating-out menus. Storage 50 is formed of a volatile memory or a nonvolatile memory.

Acquisition unit 52 is a processing unit that acquires user's conditions for the eating-out menu. Acquisition unit 52 acquires conditions including information on the dietary restrictions designated by the user, information on illness from which the user suffers, and the like.

Based on the calories or the nutrients, which are output by analyzer 10, and on eating-out data 51 including the ingestion of the food or the consumption of the calorie when eating the eating-out menu, generator 20B generates information on an eating-out menu which the user should eat. The information on such eating-out menus includes information itself stored in eating-out data 51 and information formed by processing the information stored in eating-out data 51. Specifically, the information on the eating-out menu includes, for example names and maps of the restaurants, names, calories and nutrients of the eating-out menus, and the like. Generator 20B is one of the specific configurations of generator 20.

Presentation unit 30 presents the information generated by generator 20B.

FIG. 10 is an explanatory view showing an example of eating-out data 51 in the present exemplary embodiment.

As shown in FIG. 10, eating-out data 51 includes the respective pieces of information on the names of the restaurants, the eating-out menus, the calories, the positions and the maps. For example, the eating-out data shown in FIG. 10 indicates that the name of the restaurant is “A”, that the position of the restaurant is (x1, y1) (for example, longitude is x1, latitude is y1), and the restaurant represented by map data “map_a.gif” provides the eating-out menu “tofu hamburger steak” with a calorie of 300 kcal. Note that eating-out data 51 may further include information on the nutrients of the eating-out menus.

Generator 20B acquires the information on the eating-out menu, which satisfies the conditions acquired by acquisition unit 52, based on the user's conditions for the eating-out menu (which are an upper calorie limit, a nutrient to be taken in, a nutrient not to be taken in, and the like), the user's conditions having been acquired by acquisition unit 52, and based on the information on the calories or the nutrients, which are contained in the food taken in by the user, the information having been analyzed by analyzer 10.

Note that generator 20B may generate, as the information on the eating-out menu, information indicating a moving path from a current location to the restaurant based on map information, information indicating consumed calories when the user moves along the moving path, and the like. When selecting the eating-out menu that satisfies the above-described conditions, generator 20B may determine the conditions by subtracting, from the calories of the eating-out menu, an amount of the consumed calories when the user moves to the restaurant. At this time, when the calories of the eating-out menu exceed such an upper limit of the calories capable of being taken in by the user, generator 20B intentionally lengthens the moving path (for example, causes the user to take a roundabout course, to travel on foot for a long time, and so on), and thereby may cause the calories, which are obtained by subtracting the consumed calories required for the movement from the calories of the eating-out menu, to satisfy the above-described conditions.

A description will be given below of an example of presenting the information generated by generator 20B.

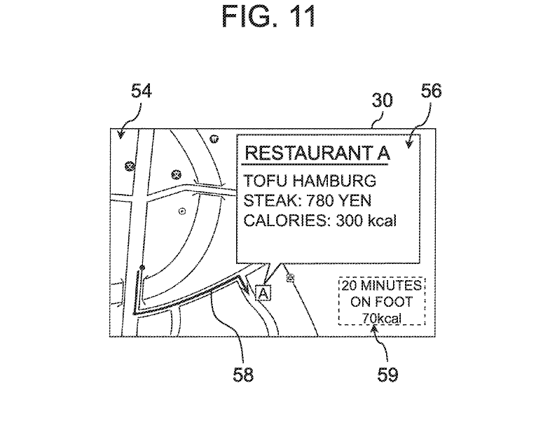

FIG. 11 is an explanatory view showing an example of the information to be presented by presentation unit 30 in the present exemplary embodiment. As an example, this information is displayed on a display as presentation unit 30.

FIG. 11 is an image including information 56, moving path 58 and information 59 together with map 54. Information 56 relates to the eating-out menu “tofu hamburger steak” offered at restaurant A. Moving path 58 is a path from a current location to restaurant A. Information 59 indicates a time and consumed calories when the user moves along moving path 58.

As described above, the information on the eating-out menu offered by the restaurant is provided. Therefore, even those who need the dietary restrictions are facilitated to determine the restaurant and the menu, and are consequently facilitated to each out. Also for the restaurants, there are advantages that the number of customers increases and that the sales increase.

Note that a sightseeing area that exists near moving path 58 may be presented on map 54, and the consumed calories during the movement to the restaurant may be increased when the user selects to drop by the sightseeing area. In this way, in the determination of the conditions by generator 20B, more eating-out menus can be allowed to satisfy the conditions.

Note that data of the calories and the nutrients, which is included in eating-out data 51, may be data input by a worker of the restaurant by using analyzer 10, or may be data obtained by another measurement method or a calculation method, followed by input, or may be data input by a customer at the restaurant by using analyzer 10.

As described above, information presentation system 3 of the present exemplary embodiment presents the calories and nutrients of the menu offered by the restaurant. Therefore, not only the average persons but also persons who need the dietary restrictions are facilitated to determine the restaurant and the menu. Hence, the display of the analysis result by the analyzer can be improved, and the convenience for the user can be enhanced. Moreover, the user is facilitated to eat out, and therefore, for the restaurants, there are advantages that the number of customers increases and that the sales increase.

### Fourth Exemplary Embodiment

In the present exemplary embodiment, a description will be given of an information presentation system that improves the display of the analysis result of the food analyzer, provides a recipe of a recommended meal menu at home, and orders ingredients for cooking the meal menu.

The recipe of the meal menu is provided by the information presentation system, and the ingredients are ordered thereby, whereby the user gets an advantage of being capable of easily acquiring the ingredients and the recipe, which are required to cook the meal menu. For sellers of the ingredients, there is an advantage that sales amounts of the ingredients increase.

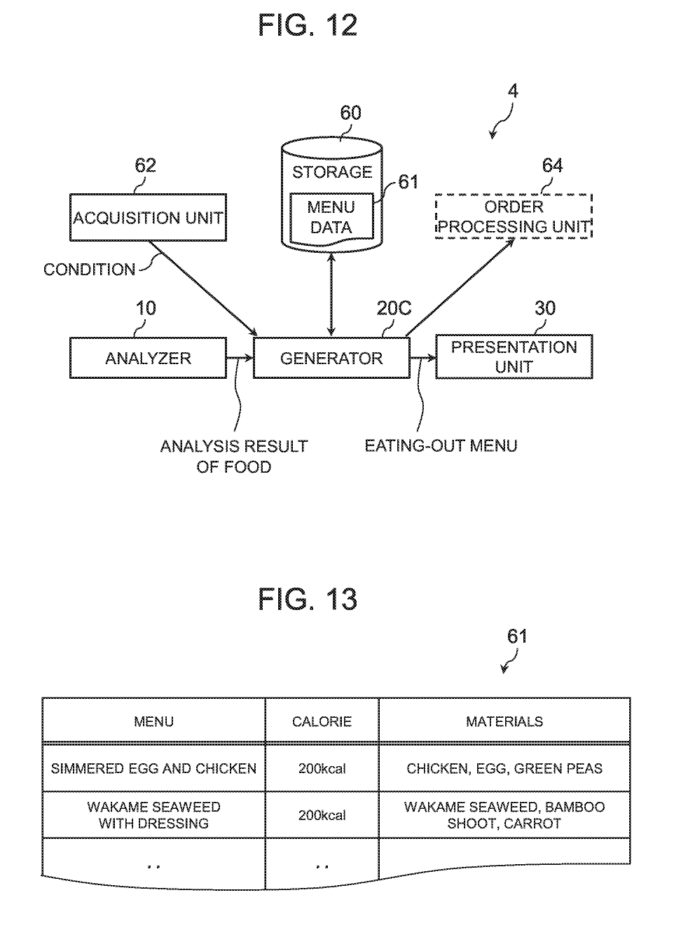

FIG. 12 is a block diagram showing a configuration of information presentation system 4 in the present exemplary embodiment.

As shown in FIG. 12, information presentation system 4 includes analyzer 10, generator 20C, presentation unit 30, storage 60 and acquisition unit 62. Moreover, information presentation system 4 may further include order processing unit 64.

Analyzer 10 is a food analyzer that makes an analysis for food taken in by the user and outputs calories or nutrients, which are contained in the food, as an analysis value. This analyzer 10 is similar to analyzer 10 in the first exemplary embodiment. Analyzer 10 analyzes at least one of the calories or the nutrients, which the user takes in from meals every day.

Storage 60 is a storage device that stores menu data 61 including one or more meal menus. Storage 60 is formed of a volatile memory or a nonvolatile memory.

Acquisition unit 62 is a processing unit that acquires user's conditions for the cooking menu. Acquisition unit 62 acquires such conditions as a meal preference designated by the user and the upper calorie limit of the meal.

Based on the calories or the nutrients, which are output by analyzer 10, and on cooking data including calories or nutrients of ingredients required to cook a plurality of the cooking menus, generator 20C generates information on a cooking menu which the user should eat. Here, the information on the cooking menu described above includes at least the ingredients required to cook the cooking menu. With reference to menu data 61, generator 20C selects the cooking menu that satisfies the conditions acquired by acquisition unit 62, and generates information on the selected cooking menu.

Generator 20C is one of the specific configurations of generator 20.

Presentation unit 30 presents the information generated by generator 20C.

Order processing unit 64 is a processing unit that processing for ordering the ingredients, which are required to cook the cooking menu and are included in the information on the cooking menu. Here, this information is generated by generator 20C. Order processing unit 64 is connected to external devices of the ingredient sellers by a communication line, and executes the processing for ordering the above-described ingredients. Note that order processing unit 64 may perform processing for ordering a cooking menu, which is already cooked, in place of the processing for ordering the ingredients required to cook the cooking menu.

FIG. 13 is an explanatory view showing an example of menu data 61 in the present exemplary embodiment.

Menu data 61 includes the respective pieces of the information on the menus, the calories, and the ingredients. For example, menu data 61 shown in FIG. 13 indicates that calories of a menu “simmered egg and chicken” are 200 kcal, and that this menu is cooked by using ingredients “chicken, egg and green peas”. Note that menu data 61 may further include information on the nutrients of the cooking menu.

With reference to menu data 61, generator 20C selects the cooking menu that satisfies the conditions acquired by acquisition unit 62, and generates the information on the selected cooking menu.

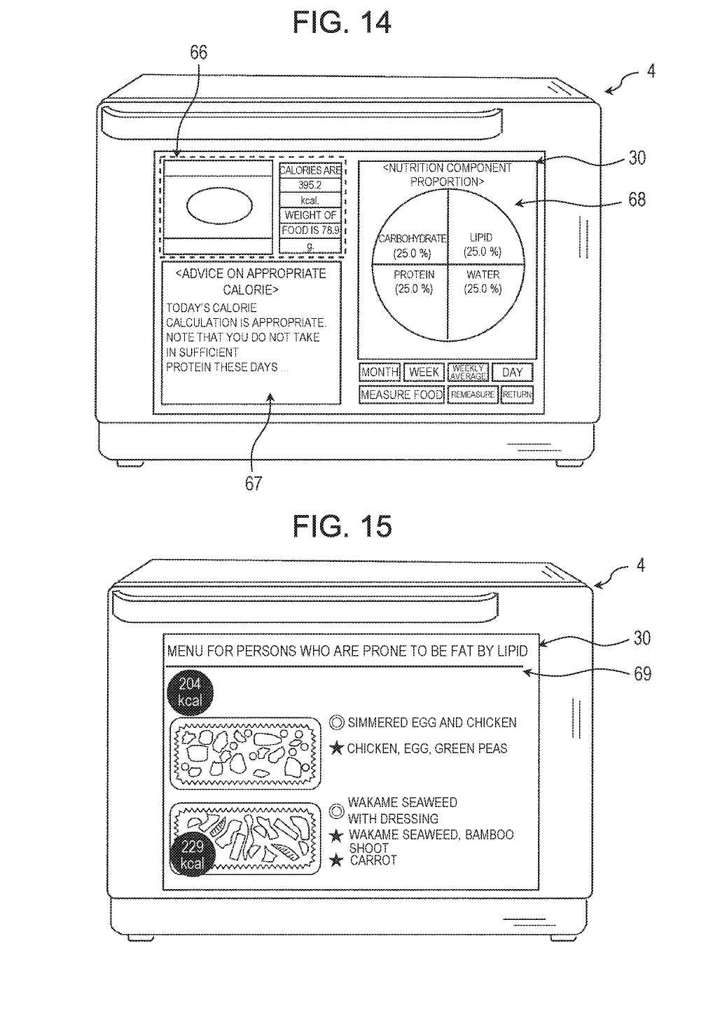

FIG. 14 is an explanatory view showing a first example of the presentation by information presentation system 4 in the present exemplary embodiment. Note that FIG. 14 shows an example in which a display panel as presentation unit 30 is disposed on a front panel of analyzer 10. As described above, presentation unit 30 is disposed on the front panel of analyzer 10, whereby the user gets an advantage of being capable of easily recognizing visually and receiving the information presented by information presentation system 4.

On presentation unit 30 shown in FIG. 14, there are displayed: information 66 indicating calories and weights of food, which are analysis result of the food by analyzer 10; message 67 related to the analysis result of the food; and information 68 indicating nutrition component proportions as the analysis result of the food.

The user analyzes daily meal menus by analyzer 10 and visually recognizes an image of presentation unit 30, which is shown in FIG. 14.

FIG. 15 is an explanatory view showing a second example of the presentation by information presentation system 4 in the present exemplary embodiment.

Information 69, which is related to the cooking menus and is generated by generator 20C, is presented on presentation unit 30 shown in FIG. 15. As an example, information 69 is information including meal menus oriented for persons who are prone to be fat by lipid. When it is determined that the user has a constitutional predisposition to be prone to be fat by lipid based on the daily meals of the user, presentation unit 30 presents information 69, and can thereby present an appropriate menu to the user. When the user selects the menu to be cooked from among the menus included in information 69, order processing for ingredients of the selected menu is carried out by order processing unit 64.

As described above, information presentation system 4 of the present exemplary embodiment provides the recipe of the meal menu, which the user should eat, based on the foods taken in by the user. Hence, the user can easily acquire the recipe required to cook the meal menu.

Moreover, based on the foods taken in by the user, information presentation system 4 provides the recipe of the meal menu, which the user should eat, and in addition, orders the ingredients required to cook the meal menu. Hence, the user can easily acquire the recipe required to cook the meal menu, and in addition, the ingredients required to cook the meal menu.

### Fifth Exemplary Embodiment

In the present exemplary embodiment, a description will be given of an information presentation system that improves the display of the analysis result of the food analyzer and provides an exercise menu and a meal menu, which are recommended for managing a body shape of the user, and so on.

The user exercises in accordance with the exercise menu provided by the information presentation system and has a meal in accordance with the meal menu presented thereby, and can thereby bring the body shape of the user him/herself closer to a target body shape, or can maintain the target body shape.

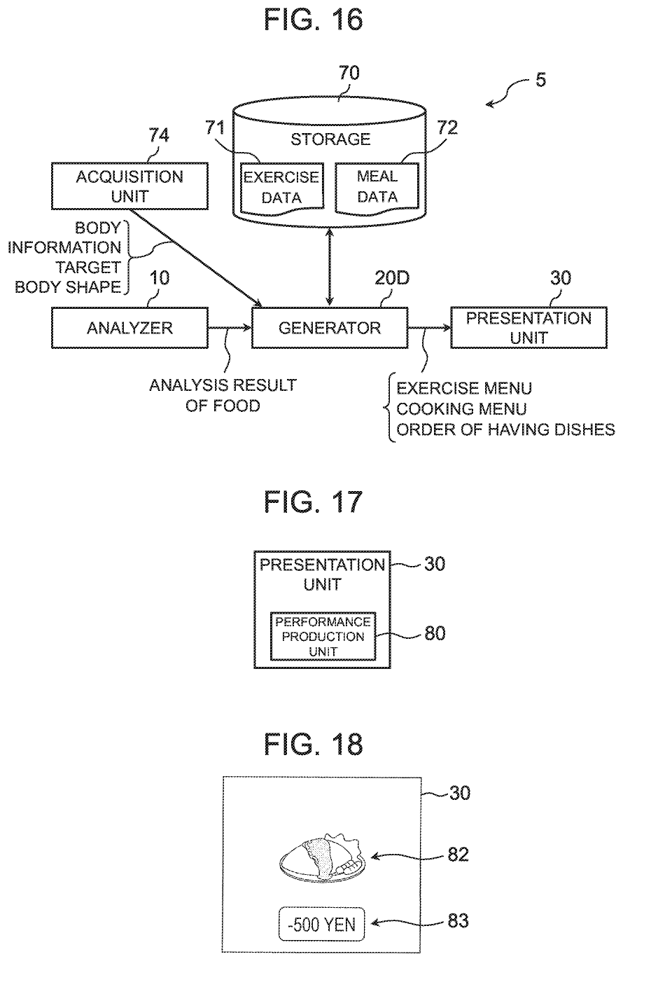

FIG. 16 is a block diagram showing a configuration of information presentation system 5 in the present exemplary embodiment.

As shown in FIG. 16, information presentation system 5 includes analyzer 10, generator 20D, presentation unit 30, storage 70, and acquisition unit 74.

Analyzer 10 is a food analyzer that makes an analysis for food taken in by the user and outputs calories or nutrients, which are contained in the food, as an analysis value. This analyzer 10 is similar to analyzer 10 in the first exemplary embodiment. Analyzer 10 analyzes at least one of the calories or the nutrients, which the user takes in from meals every day.

Storage 70 is a storage device that stores exercise data 71 including one or more exercise menus, and meal data 72 including one or more meal menus. Storage 70 is formed of a volatile memory or a nonvolatile memory.

Acquisition unit 74 acquires body information indicating the body shape of the user and a target body shape (body shape which the user takes as a target). For example, the body information includes information such as height, weight, a body fat percentage and the like. Moreover, the target body shape includes, for example, an athlete (power) type, an athlete (stamina) type, a slim type, and the like.

Exercise data 71 is data in which the target body shape is associated with the exercise menu suitable for achieving the target body shape (or maintaining the target body shape).

Meal data 72 is data in which the target body shape is associated with the meal menu suitable for achieving the target body shape (or maintaining the target body shape). Note that meal data 72 may include information that specifies an order of having the respective meal menus. For example, meal data 72 includes information that specifies the order of having the respective meal menus so that a meal menu containing a relatively small amount of carbohydrate is eaten first, and so that a meal menu containing a relatively large amount of carbohydrate is eaten later.

Based on a current body shape and target body shape of the user, which are acquired by acquisition unit 74, and based on the calories or the nutrients, which are output by analyzer 10, generator 20D generates information on the exercise menu which the user should do in order to achieve the target body shape, or information on the food which the user should take in. Specifically, generator 20D acquires body information indicating the current body shape of the user, the target body shape, and the calories or nutrients of the food taken in by the user. Then, with reference to exercise data 71, generator 20D determines the exercise menu required for the user to achieve the target body shape. Moreover, with reference to meal data 72, generator 20D determines the meal menu required for the user to achieve the target body shape. Note that, based on meal data 72, generator 20D may determine information indicating the order of having dishes in the meal menu. Note that generator 20D is one of the specific configurations of generator 20.

Presentation unit 30 presents the information generated by generator 20D. Specifically, presentation unit 30 acquires and presents the information, which indicates the exercise menu and the cooking menu and is generated by generator 20D. Note that, when generator 20D generates the information on the order of having dishes, presentation unit 30 acquires and presents the information on the order of having dishes.

Note that acquisition unit 74 may further acquire meal menus desired by the user. Then, generator 20D may further consider a history of such analysis results of analyzer 10 and the above-described meal menus acquired by acquisition unit 74, and may generate information on the exercise menu which the user should do in order to achieve the target body shape, or information on the food which the user should take in.

As described above, information presentation system 5 of the present exemplary embodiment provides the exercise menu which the user should do, or the cooking menu which the user should eat. The user exercises in accordance with the provided exercise menu or has a meal in accordance with the presented cooking menu, and can thereby bring the body shape of the user him/herself closer to the target body shape, or can maintain the target body shape.

### Sixth Exemplary Embodiment

In the present exemplary embodiment, a description will be given of a technique for improving the display of the analysis result of the food analyzer and assisting the user to continuously use the information presentation system.

The information provided by analyzer 10 according to each of the exemplary embodiments described above is a numeric value indicating the calories or nutrients of the food. Moreover, the information provided by the information presentation system in each of the exemplary embodiments described above is information on the ingestion of the food by the user or the consumption of the calories by the user, the information including the meal menu or the exercise menu. When the user repeatedly receives the provision of these pieces of information, the user loses interest in the provided information, or is tired therewith, and may thereby stop using the information presentation system.

An information presentation system according to the present exemplary embodiment is able to prevent the user from stopping the use of the information presentation system.

A configuration of the information presentation system in the present exemplary embodiment may be any of the respective exemplary embodiments described above. Here, a description will be given on the assumption that the information presentation system of the first exemplary embodiment is used.

FIG. 17 is a block diagram showing a configuration of presentation unit 30 in the present exemplary embodiment.

As shown in FIG. 17, presentation unit 30 includes performance production unit 80.

Performance production unit 80 is a processing unit that generates information having a performance production effect for the user based on the information presented by presentation unit 30. The information generated by performance production unit 80 is presented by presentation unit 30. The performance production effect for the user refers to an effect that causes the user to feel such that the information is fresh or interesting when the user visually recognizes the information.

Performance production unit 80 acquires the information acquired from generator 20 by presentation unit 30, and converts the acquired information into different information. For example, upon acquiring the information on the numeric value indicating the calories of the food from analyzer 10, performance production unit 80 converts the acquired information on the numeric value indicating the calories into information indicating fluctuations of an insurance fee or a medical bill in future, and thereby performs the performance production. Moreover, performance production unit 80 may carry out performance production for a game in which benefit information (so-called points) equivalent to a predetermined amount of monetary values is imparted to the user with a predetermined probability when the user analyzes the food, or may carry out performance production in which benefit information having a monetary value equivalent to a small amount is imparted to the user when the user analyzes the food. Note that the above-described benefit information may be one (so-called coupon and service ticket) exchangeable with a part or all of goods or services. Moreover, in place of the above-described benefit information, there may be adopted information that does not have a direct monetary value. For example, this information is a picture, a photograph or an illustration, a character, sentences, a word, an image, music and the like.

Presentation unit 30 presents the information which performance production unit 80 generates by the conversion.

FIG. 18 is a block diagram showing a first example of the image to be presented by presentation unit 30 in the present exemplary embodiment. The image shown in FIG. 18 includes: image 82 showing food to be analyzed; and image 83 showing the fluctuations of the insurance fee or the medical bill in the future.

When the information (for example, 500 kcal) on the numeric value indicating the calories of the food to be analyzed by analyzer 10 is output from analyzer 10, performance production unit 80 converts the information on the numeric value indicating the calories into the information (−500 yens) indicating the fluctuations of the insurance fee or the medical bill in the future. As a calculation method for converting the calories into the information indicating the fluctuations of the insurance fee or the medical bill in the future, an arbitrary method is adoptable. For example, here is supposed a case, in which the user gets sick when the user takes in the above-described food excessively. Then, the above-described information is obtained by calculating an increment of the insurance fee when the user gets sick as described above, inverting a sign of the increment, and thereafter, dividing the increment by a period while the insurance fee increases. Moreover, the above-described information can also be obtained by dividing a treatment pay, which is required until the user is fully recovered when the user gets sick, by a period until the user is fully recovered.

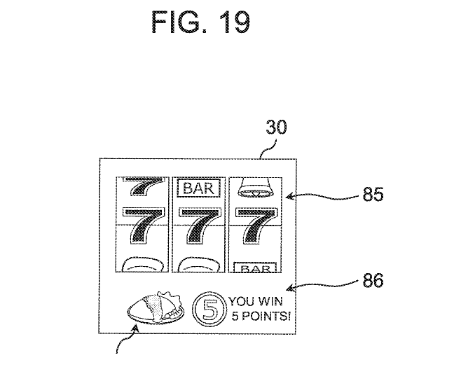

FIG. 19 is a block diagram showing a second example of the image to be presented by presentation unit 30 in the present exemplary embodiment. The image shown in FIG. 19 includes: image 82 showing the food to be analyzed; image 85 related the performance production of the game; and image 86 showing that the benefit information is imparted.

When presentation unit 30 acquires the information from generator 20, and converts and presents the information, then performance production unit 80 presents image 85 related to the game (for example, a slot game). The user plays the game while visually recognizing image 85, and can thereby obtain the information presented by the information presentation system while enjoying the game.

Moreover, when presentation unit 30 acquires the information from generator 20, and converts and presents the information, performance production unit 80 imparts the benefit information to the user. As the benefit information, information (so-called points) equivalent to the predetermined amount of monetary value in a specific store is adoptable. For example, performance production unit 80 generates benefit information different depending on a specific ratio or random element. The specific ratio is, for example, a ratio of once per predetermined number of times. Note that the benefit information may vary in response to a result of the game (for example, may be set relatively large only when a hit comes out), or may be fixed irrespective of the result of the game. Moreover, the benefit information is imparted to the user who provides the calories or nutrients of the food to the recipe site in the second exemplary embodiment, and the benefit information is imparted to the user who provides the calories or nutrients of the food of the eating-out menu in the third exemplary embodiment, whereby the user can be given an incentive to provide the information.

Note that, in a case of supposing that the food analysis by analyzer 10 is carried out at a relatively high frequency (for example, every day or every meal) (for example, this case applies to the third, fourth and fifth exemplary embodiments), if the analysis by analyzer 10 is not carried out at the supposed frequency, then performance production unit 80 may present information indicating that the already acquired benefit information will be lost. In this way, the user is given a feeling that he/she loses unless analyzer 10 is used, and as a reflection effect of being given the feeling, the user can be made to use analyzer 10 at a relatively high frequency.

Moreover, when a plurality of users use information presentation system 1, performance production unit 80 may also present an analysis result or presentation information about a single user to other users, or may share the analysis result or the presentation information with the other users. At this time, users at the same or similar age or users having the same or similar target may be grouped, and the analysis result or the presentation information may be presented or shared in the group. In this way, the sharing of the information among the users advances, trends of other users appear, and competitive consciousness or companionship consciousness are developed, whereby the users can use information presentation system 1 actively.

In such a manner, each of the users uses the benefit information, which is obtained every time of analyzing the food, at the specific shop, and can thereby carry out a substantial discount purchase, or can enjoy the obtained information itself. Moreover, the user can grasp the trends of the other users, and can tackle the analysis of the food more actively. In such a manner, the user uses the information presentation system actively or while feeling that he/she has gained benefit. Therefore, it is made possible for the user to use the information presentation system continuously.

As described above, the information presentation system of the present exemplary embodiment presents the information produced so that the user has some kind of feeling. This information is generated based on the information on the numeric value indicating the calories or nutrients of the food, the ingestion of the food by the user, and the consumption of the calories by the user. In this way, the user is prevented from losing interest in the provided information, and the user can obtain the information by continuously using the information presentation system.

Moreover, the information presentation system presents the benefit information at a predetermined ratio. When the information is provided from the information presentation system, the user sometimes obtains the benefit information, and sometimes does not obtain the benefit information. In this way, the user uses the information presentation system while expecting that he/she can obtain the benefit information. In this way, the user is prevented from losing interest in the provided information, and the user can obtain the information by continuously using the information presentation system.

Note that, in each of the exemplary embodiments described above, the respective constituents may be composed of dedicated hardware, or may be implemented by executing a software program suitable for each of the constituents. The respective constituents may be implemented in such a manner that a program execution unit such as a CPU and a processor read out and executes the software programs recorded in a recording medium such as a hard disk and a semiconductor memory. Here, the software that implements the information presentation system of each of the exemplary embodiments described above and the like is a program as described below.

That is, this program causes a computer to execute a control method for an information presentation system, the control method including: an analysis step of analyzing food and outputting an analysis value indicating a result of the analysis; a generation step of generating information on ingestion of the food by a user or consumption of calories by the user based on the analysis value output in the analysis step; and a presentation step of presenting the information generated in the generation step.

Note that, in the above description, the description “A or B” may include “at least one of A and B”.

As above, the description has been given of the information presentation systems according to one or plural aspects based on the exemplary embodiments; however, the present disclosure is not limited to the exemplary embodiments. As long as such forms as follows do not depart from the spirit of the present disclosure, forms, which are obtained by applying a variety of modifications conceived by those skilled in the art to the present exemplary embodiment, and forms, which are constructed by combining the constituents in the different the present exemplary embodiments, may be included within the range of the one or plural aspects.

The present disclosure is applicable to the information presentation system that presents the information output by the food analyzer that presents the calories or nutrients of the food.

What is claimed is:

1. An information presentation system comprising:
an analyzer that makes an analysis of food and outputs an analysis value indicating a result of the analysis;
a generator that generates information on at least one of food ingestion by a user and calorie consumption by the user, based on the analysis value output by the analyzer; and
a presentation unit that presents the information generated by the generator.

2. The information presentation system according to claim 1, wherein
the analyzer analyzes food cooked in accordance with a recipe of a cooking menu, and outputs, as the analysis value, at least one of calories and nutrients contained in the food,
the generator generates information indicating at least one of the calories and the nutrients of the cooking menu, based on at least one of the calories and the nutrients contained in the food and output by the analyzer, and
the presentation unit presents, together with the recipe, the information indicating at least one of the calories and the nutrients, the information being generated by the generator.

3. The information presentation system according to claim 1, further comprising a storage that stores recipes of one or more cooking menus,
wherein
the analyzer analyzes food cooked in accordance with the recipe of a cooking menu included in the one or more cooking menus, and outputs, as the analysis value, at least one of calories and nutrients contained in the food,
the generator generates information indicating at least one of the calories and the nutrients of the cooking menu based on at least one of the calories and the nutrients contained in the food and output by the analyzer, and causes the storage to store the information generated, and
the presentation unit presents, together with a recipe of a target cooking menu searched by the user among the one or more cooking menus stored in the storage, information indicating at least one of the calories and the nutrients of the target cooking menu stored in the storage.

4. The information presentation system according to claim 1, wherein
the analyzer analyzes food taken in by the user, and outputs, as the analysis value, at least one of calories and nutrients contained in the food, and
the generator generates information on an eating-out menu that the user should eat, based on at least one of the calories and the nutrients output by the analyzer, and on eating-out menu data including at least one of ingestion of food and consumption of calories when the user eats an eating-out menu.

5. The information presentation system according to claim 1, wherein
the analyzer analyzes food taken in by the user, and outputs, as the analysis value, at least one of calories and nutrients contained in the food,
the generator generates information on a cooking menu that the user should eat, based on at least one of the calories and the nutrients output by the analyzer, and on cooking data including at least one of calories and nutrients of ingredients required to cook a plurality of the cooking menus, and
the information on the cooking menu includes at least the ingredients required to cook the cooking menu.

6. The information presentation system according to claim 5, further comprising an order processing unit that performs order processing for at least one of the ingredients required to cook the cooking menu and the cooking menu already cooked, the ingredients being included in the information on the cooking menu, the information being generated by the generator.

7. The information presentation system according to claim 1, wherein
the analyzer analyzes food taken in by the user, and outputs, as the analysis value, at least one of calories and nutrients contained in the food, and
the generator generates at least one of information on an exercise menu that the user should do in order to achieve a target body shape, and information on food that the user should take in, based on a current body shape and target body shape of the user and on at least one of the calories and the nutrients output by the analyzer.

8. The information presentation system according to claim 1, wherein the presentation unit
includes a performance production unit that generates information having a performance production effect for the user based on the information indicating at least one of the calories and the nutrients, the information being generated by the generator, and
presents the information having the performance production effect for the user, the information being generated by the performance production unit.

9. The information presentation system according to claim 8, wherein
the performance production unit generates benefit information different depending on at least one of a specific ratio and a random element when the presentation unit presents the information having the performance production effect for the user, and
the presentation unit presents the benefit information.

10. The information presentation system according to claim 1, wherein the analyzer analyzes at least one of components of the food, the components including calories, carbohydrate, lipid, protein, dietary fiber, and glucide.

11. A method for controlling an information presentation system, the method comprising:
an analysis step of making an analysis for food and outputs an analysis value indicating a result of the analysis;
a generation step of generating information on at least one of food ingestion by a user and calorie consumption by the user based on the analysis value output in the analysis step; and
a presentation step of presenting the information generated in the generation step.
